(19) United States
(12) Patent Application Publication
(10) Pub. No.: US 2008/0188538 A1
(43) Pub. Date: Aug. 7, 2008

(54) METHODS OF LOWERING GLUCOSE LEVELS

(76) Inventors: Hertzel GERSTEIN, Hamilton (CA); Salim YUSUF, Carlisle (CA)

Correspondence Address: JONES DAY, 222 EAST 41ST ST, NEW YORK, NY, 10017, US

(21) Appl. No.: 11/846,310

(22) Filed: Aug. 28, 2007

Related U.S. Application Data
(60) Provisional application No. 60/840,850, filed on Aug. 28, 2006.

(51) Int. Cl. A61K 31/403; A61P 3/10

(52) U.S. Cl. 514/412

(57) ABSTRACT

The present invention is directed to methods of lowering glucose levels in a patient. More specifically, the present invention is directed toward methods of lowering glucose levels comprising administering a therapeutically effective amount of ramipril to a patient in need thereof.

This application claims the benefit of provisional application 60/840,850, filed on Aug. 28, 2006, which is incorporated herein by reference in its entirety.

### FIELD OF THE INVENTION

The present invention is directed to methods of lowering glucose levels in a patient. More specifically, the present invention is directed toward methods of lowering glucose levels comprising administering a therapeutically effective amount of ramipril to a patient in need thereof.

### BACKGROUND

Diabetes mellitus is a group of metabolic diseases that includes type 2 diabetes and is characterized by high blood sugar (glucose) levels, which result from defects in insulin secretion, action, or both. The prevalence of diagnosed diabetes mellitus, including type 2 diabetes, is currently increasing. Affected individuals have a high risk of blindness, renal failure, amputations, myocardial infarction and stroke. Additionally, diabetes mellitus also increases the risk of cardiovascular (CV) death 2-3 fold in men and 3-4 fold in women. Such disorders associated with diabetes mellitus account for annual health care expenditures that exceeded $130 billion dollars in 2002 in the United States alone. Diabetologia, Vol. 47, 1519-1527 (2004).

Much energy has been directed to researching ways of reducing or preventing the occurrence of the disorders associated with diabetes, as well as, preventing the development of diabetes. Upon re-evaluation of the data generated from the Heart Outcomes Prevention (HOPE) Study, which was conducted to show that the ACE inhibitor ramipril was successful in reducing cardiovascular events, the results also implied that ramipril could possibly prevent the development of diabetes in patients 55 years of age or older and who have evidence of prior coronary artery disease, stroke, or peripheral vascular disease and one additional risk factor (current or previous hypertension, elevated total cholesterol, low HDL cholesterol, current cigarette smoking, known microalbuminuria or previous vascular disease). The HOPE study was originally scheduled to be conducted over a period of 3.5 years but was extended to 5 years. The HOPE study and such data re-analysis are described in PCT Application WO/0115674, European Patent EP 1437131 and U.S. Application Pub. Nos. 2004/0087645 and 2005/0065203.

PCT Application WO/0115674 discloses the use of ramipril to prevent or reduce the onset of diabetes in a patient who is at high risk for cardiovascular events due to a history of previous ischaemic heart disease, stroke or peripheral arterial disease.

European Patent EP 1437131 discloses the use of ramipril to prevent or reduce the onset of diabetes in a patient who is at high risk for cardiovascular events due to a history of previous ischaemic heart disease, stroke, peripheral arterial disease but who have no preexisting congestive heart failure.

U.S. Application Pub. No. 2004/0087645 discloses the use of ramipril to prevent cardiovascular events in a patient with prior coronary artery disease, stroke, peripheral vascular disease or diabetes plus at least one other risk factor.

Additionally, U.S. Application Pub. No. 2005/0065203 discloses the use of ramipril to prevent the development of diabetes in a patient who has evidence of vascular disease and at least one other risk factor.

However, recent findings have shown that the risk of an individual being affected by at least some of these disorders such as, cardiovascular events, blindness, renal failure, amputations, myocardial infarction or stroke, is elevated, not only when the individual's blood glucose levels reach diabetic glucose levels, but also at glucose levels well below the diabetes cut-off. Individuals with increased glucose levels that are below diabetic glucose levels are usually diagnosed with impaired glucose tolerance (IGT) or impaired fasting glucose (IFG).

People with IGT and/or IFG have blood glucose levels that are higher than normal but lower than diabetic glucose levels. IGT is usually diagnosed using an oral glucose tolerance test (OGTT). The OGTT includes measuring blood glucose levels after a fast and after a glucose challenge. The OGTT consists of first measuring an individual's glucose level after an 8 to 12 hour fast. The individual then drinks a glucose-containing solution and, after 2 hours, the individual's blood glucose level is measured again. If the individual's blood glucose remains below 140 mg/dl, the individual is considered to have normal glucose tolerance. If the individual's blood glucose rises to between 140 mg/dl and 199 mg/dl, the individual is considered to have impaired glucose tolerance (IGT). If the individual's blood glucose rises above 200 mg/dl, the individual is considered to be diabetic.

IFG is usually diagnosed using a fasting blood glucose test, which consists of measuring an individual's blood glucose level after an 8 to 12 hour fast. After completing the fasting glucose test, if the individual's blood glucose level is below 110 mg/dl the individual is considered to have normal blood glucose levels. If the individual's blood glucose level is between 110 mg/dl and 125 mg/dl, the individual is considered to have impaired fasting glucose. If the individual's blood glucose level rises to 126 mg/dl or above, the individual is considered to be diabetic.

The prevalence of both IGT and IFG rises with age and varies with ethnicity. For example, the prevalence rates of IGT in Americans aged 40-49, 50-59 and 60-74 are 11.9%, 14.3% and 20.7% respectively. The rates of IGT and IFG among 40-74 year olds are 15.8% (15.2% of men and 16.4% of women) and 9.7% respectively. When examined by ethnicity, the rates for non-Hispanic whites, non-Hispanic blacks, and Hispanic Mexican Americans are 15.3%, 14% and 20.2% respectively for IGT, and 9.5%, 9.4% and 12.2% respectively for IFG. Within Canada, the IGT rates for European, South Asian, Chinese and aboriginals range from 8-19%.

Lowering glucose levels of individuals can prevent the increase of glucose levels to levels that correspond with IGT, IFG or diabetes, as well as, reduce the risk of developing disorders associated with increased glucose levels. Therefore, there exists a need for methods of lowering glucose levels in individuals with elevated glucose levels. Additionally, there exists a need for methods of lowering glucose levels in individuals with elevated glucose levels that also reduce the risk of developing health disorders associated with increased glucose levels.

### SUMMARY OF THE INVENTION

The inventors have found that glucose levels can be lowered by administering a therapeutically effective amount of ramipril to patients in need thereof. For example, the inventors have found that by administering ramipril to individuals with increased glucose levels, such as individuals who have been diagnosed with impaired glucose tolerance or impaired fasting glucose, or both impaired glucose tolerance and impaired fasting glucose, glucose levels can be lowered to normal glucose levels. Additionally, by administering ramipril to individuals with increased glucose levels, such as individuals who have been diagnosed with diabetes, glucose levels can be lowered to below diabetic levels such as, glucose levels corresponding to impaired glucose tolerance and impaired fasting glucose. Also, by administering ramipril to individuals with increased glucose levels, such as individuals who have been diagnosed with diabetes, glucose levels can be lowered to normal glucose levels. Also, in addition to lowering glucose levels of individuals with impaired glucose tolerance, impaired fasting glucose or diabetes, the administration of ramipril can reduce the occurrence of other disorders associated with elevated glucose levels, including, cardiovascular events, blindness, renal failure, amputations, myocardial infarction and strokes.

In one embodiment, described herein are methods of lowering glucose levels by administering a therapeutically effective amount of ramipril or a pharmaceutically acceptable salt thereof to a patient in need thereof. For example, described herein are methods of lowering glucose levels in a patient by administering to a patient diagnosed with dysglycemia, a therapeutically effective amount of ramipril or a pharmaceutically acceptable salt thereof for a sufficient period of time to reduce the glucose levels in said patient. For all methods described herein, patients with dysglycemia include patients that have been diagnosed with diabetes, impaired glucose tolerance, impaired fasting glucose, or both impaired glucose tolerance and impaired fasting glucose. In certain embodiments, such dysglycemic patients can have no history of cardiovascular events. In other embodiments, such dysglycemic patients include patients with a history of cardiovascular events.

For example, described herein are methods of lowering glucose levels in a patient by administering to a patient with impaired glucose tolerance or impaired fasting glucose, or both impaired glucose tolerance and impaired fasting glucose, a therapeutically effective amount of ramipril or a pharmaceutically acceptable salt thereof for a sufficient period of time to reduce the glucose levels in said patient.

Additionally, described herein are methods of lowering glucose levels in a patient by administering to a patient with diabetes, a therapeutically effective amount of ramipril or a pharmaceutically acceptable salt thereof for a sufficient period of time to reduce the glucose levels in said patient. Also described herein are methods of lowering glucose levels in a patient by administering to a patient with diabetes and no history of cardiovascular disease, a therapeutically effective amount of ramipril or a pharmaceutically acceptable salt thereof for a sufficient period of time to reduce the glucose levels in said patient.

In another embodiment, described herein are methods of preventing or reducing the frequency of a disorder associated with elevated glucose levels by administering to a patient diagnosed with dysglycemia, a therapeutically effective amount of ramipril or a pharmaceutically acceptable salt thereof for a sufficient period of time to prevent or reduce the frequency of said disorder. For example, described herein are methods of preventing or reducing the frequency of a disorder associated with elevated glucose levels by administering to a patient with impaired glucose tolerance or impaired fasting glucose, or both impaired glucose tolerance and impaired fasting glucose, a therapeutically effective amount of ramipril or a pharmaceutically acceptable salt thereof for a sufficient period of time to prevent or reduce the frequency of said disorder.

Additionally, described herein are methods of preventing or reducing the frequency of a disorder associated with elevated glucose levels by administering to a patient with diabetes, a therapeutically effective amount of ramipril or a pharmaceutically acceptable salt thereof for a sufficient period of time to reduce the glucose levels in said patient. Also described herein are methods of preventing or reducing the frequency of a disorder associated with elevated glucose levels by administering to a patient with diabetes and no history of cardiovascular disease, a therapeutically effective amount of ramipril or a pharmaceutically acceptable salt thereof for a sufficient period of time to reduce the glucose levels in said patient.

In another embodiment, described herein are methods of lowering alanine aminotransferase (ALT) levels in a patient by administering to a patient with dysglycemia, a therapeutically effective amount of ramipril or a pharmaceutically acceptable salt thereof for a sufficient period of time to reduce ALT levels in said patient. ALT is a transaminase enzyme that is usually found in the liver and heart cells. Elevated ALT levels in the blood stream may be associated with prospective changes in liver or whole-body insulin sensitivity and/or insulin secretion. In certain embodiments, described herein are methods of lowering alanine aminotransferase (ALT) levels in a patient by administering to a patient with impaired glucose tolerance, impaired fasting glucose or both impaired glucose tolerance and impaired fasting glucose, a therapeutically effective amount of ramipril or a pharmaceutically acceptable salt thereof for a sufficient period of time to reduce ALT levels in said patient.

Additionally, described herein are methods of lowering alanine aminotransferase (ALT) levels in a patient by administering to a patient with diabetes a therapeutically effective amount of ramipril or a pharmaceutically acceptable salt thereof for a sufficient period of time to reduce the ALT levels in said patient. Also described herein are methods of lowering alanine aminotransferase (ALT) levels in a patient by administering to a patient with diabetes and no history of cardiovascular disease, a therapeutically effective amount of ramipril or a pharmaceutically acceptable salt thereof for a sufficient period of time to reduce the ALT levels in said patient.

In yet another embodiment, described herein are methods of lowering fasting plasma glucose levels comprising administering to a patient with dysglycemia, a therapeutically effective amount of ramipril for a sufficient period of time to reduce fasting plasma glucose levels in said patient. For example, described herein are methods of lowering fasting plasma glucose levels comprising administering to a patient with diabetes, impaired fasting glucose, impaired glucose tolerance or both impaired fasting glucose and impaired glucose tolerance, a therapeutically effective amount of ramipril for a sufficient period of time to reduce fasting plasma glucose levels in said patient. Such patients can have no history of cardiovascular disease.

In yet another embodiment, described herein are methods of lowering post-load glucose levels comprising administering to a patient with dysglycemia, a therapeutically effective amount of ramipril for a sufficient period of time to reduce post-load glucose levels in said patient. For example, described herein are methods of lowering post-load glucose levels comprising administering to a patient with diabetes, impaired fasting glucose, impaired glucose tolerance or both impaired fasting glucose and impaired glucose tolerance, a therapeutically effective amount of ramipril for a sufficient period of time to reduce post-load glucose levels in said patient. Such patients can have no history of cardiovascular disease.

Also, described herein are methods of preventing diabetes comprising administering to a patient diagnosed with dysglycemia, whose blood glucose levels are not elevated to a diabetic level, a therapeutically effective amount of ramipril for a period of five years or more, as well as, methods of delaying the onset of diabetes in a patient comprising administering to a patient diagnosed with dysglycemia, whose blood glucose levels are not elevated to a diabetic level, a therapeutically effective amount of ramipril for a period of five years or more.

For example, described herein are methods of preventing diabetes comprising administering to a patient diagnosed with impaired glucose tolerance or impaired fasting glucose, or both impaired glucose tolerance and impaired fasting glucose, a therapeutically effective amount of ramipril for a period of five years or more, as well as, methods of delaying the onset of diabetes in a patient comprising administering to a patient diagnosed with impaired glucose tolerance or impaired fasting glucose, or both impaired glucose tolerance and impaired fasting glucose, a therapeutically effective amount of ramipril for a period of five years or more.

### BRIEF DESCRIPTION OF THE DRAWINGS

FIG. 1 shows the percent of study participants with diabetes, IGT or IFG, or both IGT and IFG, and normal glucose levels at the end of the DREAM study.

FIG. 2 shows a Kaplan-Meier estimate of regression of individuals in the ramipril group achieving normal glucose levels in both fasting and two hour, post-load glucose levels compared to placebo.

FIG. 3 shows a Kaplan-Meier estimate of individuals who developed diabetes in the ramipril group as compared to the placebo group.

### DETAILED DESCRIPTION

### Definitions

The term “cardiovascular event(s)” as used herein encompasses any disease, illness, sickness, disorder, condition, symptom or issue involving or concerning any part or portion of the heart or blood vessels of a patient, including a human. The term “blood vessel”, as used herein, is defined to include any vessel in which blood circulates. Such cardiovascular events include, for example, arterial enlargements, arterial narrowings, peripheral artery disease, atherosclerotic cardiovascular disease, high blood pressure, angina, irregular heart rates, inappropriate rapid heart rate, inappropriate slow heart rate, angina pectoris, heart attack, myocardial infarction, transient ischemic attacks, heart enlargement, heart failure, congested heart failure, heart muscle weakness, inflammation of the heart muscle, overall heart pumping weakness, heart valve leaks, heart valve stenosis (failure-to-open fully), revascularization, ventricular arrhythmia, infection of the heart valve leaflets, heart stoppage, asymptomatic left ventricular dysfunction, cerebrovascular incidents, stroke, cardiovascular death, or ventricular tachyarrhythmia.

The terms “treat(s)”, “treated”, “treating” or “treatment” are used herein interchangeably and refer to any treatment of a disorder in a patient diagnosed or afflicted with such disorder and includes, but is not limited to: (a) caring for a patient diagnosed or afflicted with a disorder; (b) curing or healing a patient diagnosed or afflicted with a disorder; (c) causing regression of a disorder in a patient; (d) arresting further development or progression of a disorder in a patient; (e) slowing the course of a disorder in a patient; (f) relieving, improving, decreasing or stopping the symptoms of a disorder in a patient; (g) relieving, decreasing or stopping the symptoms caused by or associated with a disorder in a patient; or (h) reducing the frequency, number or severity of episodes caused by or associated with a disorder in a patient.

The terms “prevent(s)”, “prevented”, “preventing” or “prevention” are used herein interchangeably and refer to any prevention or any contribution to the prevention of a disorder in a patient or the development of a disorder if none has occurred in a patient which may be predisposed to such disorder but has not yet been afflicted with or diagnosed as having such disorder.

As used herein, the term “patient” means an animal, preferably a mammal such as a non-primate (e.g., cow, horse, sheep, pig, chicken, turkey, quail, cat, dog, mouse, rat, rabbit or guinea pig) or a primate (e.g., monkey and human), most preferably a human. Additionally, as used herein “patient”, “individual”, “subject” and “mammal”, such as human, can be used interchangeably and are not limited to individuals that are under the care of a doctor.

The phrase “therapeutically effective amount(s)”, as used herein, means any amount of a drug which, when administered to a patient in need thereof, will achieve a beneficial pharmacological effect or therapeutic improvement consistent with the objectives of the present invention without causing serious, adverse or otherwise treatment-limiting side effects (at a reasonable benefit/risk ratio), within the scope of sound medical judgment.

The term “about” as used herein means approximately or near or around. For example, when the term “about” is used in relation to a specified dosage amount or range, the term “about” indicates that the dosage amount or range specified is an approximate dosage amount or range and that it includes not only the amount or range actually specified, but those amounts or ranges that may also be safe and effective amounts that are somewhat outside the cited amount or range.

As used herein, the terms “by”, “comprising,” “comprises”, “comprised of,” “including,” “includes,” “included,” “involving,” “involves,” “involved,” and “such as” are used in their open, non-limiting sense.

It should be understood that the phrase “pharmaceutically acceptable” is used adjectivally herein to mean that the modified noun is appropriate for use in a pharmaceutical product.

The term “pharmaceutically acceptable salt” refers to a salt that retains the biological effectiveness of the free acid and/or base of the specified compound. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phylacetates, phenylpropionates, phenylbutyrates, citrates, lactates, gamma-hydroxybutyrates, glycollates, tartarates, methane-sulfonates, propanesulfonates, naphthalene-1-sulfonates, naphthalene-2-sulfonates, and mandelates. Several of the officially approved salts are listed in Remington: The Science and Practice of Pharmacy, Mack Publ. Co., Easton.

“Dysglycemia” refers to blood glucose levels that are abnormal. For example, in one embodiment the glucose levels are elevated. Dysglycemia includes diabetic and pre-diabetic glucose levels.

“Pre-diabetic glucose levels” refer to glucose levels that are higher than normal glucose levels but are not high enough to be classified as diabetic. Such pre-diabetic glucose levels include impaired fasting glucose or impaired glucose tolerance. For example pre-diabetic glucose level refers to a fasting glucose level that is above 100 mg/dl but below 125 mg/dl. Also, pre-diabetic glucose level refers to a post-load glucose level that is between 140 and 199 mg/dl.

“Fasting glucose level” refers to a patient's glucose level after a period of fasting, usually 8 to 12 hours.

“Post-load glucose level” refers to a patient's glucose level after ingesting a solution containing a predetermined amount of glucose, for example 75 g of glucose.

“Oral glucose tolerance test” or “OGTT” refers to a blood glucose test that tests glucose tolerance by measuring post-load glucose levels. The test consists of measuring plasma glucose levels after an 8 to 12 hour fast and 2 hours after ingesting 75 g of glucose. This second measurement is also referred to as a 2 hour post-load glucose level measurement.

“Impaired glucose tolerance” or “IGT” refers to a 2 hour post 75 g glucose load plasma glucose level that is between about 7.8-11.0 mmol/l (140-199 mg/dl).

“Fasting glucose test” or “fasting blood glucose test” refers to a blood glucose test that measures fasting plasma glucose levels. The test consists of measuring blood glucose levels after an 8 to 12 hour fast.

“Impaired fasting glucose” or “IFG” refers to a fasting plasma glucose level that is between 6.1-6.9 mmol/l (110-125 mg/dl).

“Normal glucose levels” refers to glucose levels that are not considered abnormal. Normal glucose levels can include normal glucose tolerance and normal fasting glucose levels.

“Normal glucose tolerance” refers to glucose levels that are below impaired glucose tolerance levels, i.e., a 2 hour post 75 g glucose load plasma glucose level that is lower than 7.8 mmol/l (140 mg/dl).

“Normal fasting glucose” refers to a fasting plasma glucose level that is below impaired fasting glucose, i.e., less than 6.1 mmol/l (110 mg/dl).

“Diabetes” or “diabetes mellitus” refers to a glucose level of ≧11.1 mmol/l [200 mg/dl] after a 2 hour oral glucose tolerance test (OGTT). “Diabetes” or “diabetes mellitus” can also refer to a fasting plasma glucose (FPG) ≧7.0 mmol/l [126 mg/dl]. However, a value below 7.0 mmol/l [126 mg/dl] does not rule out diabetes mellitus (i.e. up to 50% of people without previously diagnosed diabetes mellitus have a FPG <7.0 mmol/l [126 mg/dl]).

### Ramipril

Ramipril is an angiotensin-converting enzyme (ACE) inhibitor which lowers the production of Angiotensin II, therefore relaxing arterial muscles while at the same time enlarging the arteries, allowing the heart to pump blood easier, and increasing blood flow due to more blood being pumped into and through bigger passageways.

Ramipril, a 2-aza-bicyclo [3.3.0]-octane-3-carboxylic acid derivative with five chiral centers, and 32 different enantiomeric forms, is the prodrug to the active metabolite ramiprilat. Ramipril is converted to ramiprilat in the body by hepatic cleavage of the ester group. The chemical name of ramipril is (2S,3aS,6aS)-1[(S)-N-[(S)-1-carboxy-3-phenylpropyl]alanyl]octahydrocyclo-penta[b]pyrrole-2-carboxylic acid, 1-ethyl ester and it has the following chemical structure:

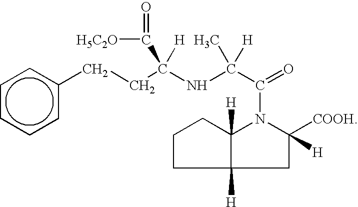

Ramipril is marketed in the United States under the brand name Altace® and abroad under the brand name Delix®. Altace® (ramipril) is supplied as hard shell capsules for oral administration containing 1.25 mg, 2.5 mg, 5 mg or 10 mg of ramipril.

Ramipril and processes for making and using ramipril are described and claimed in U.S. Pat. Nos. 4,587,258, 5,061,722 and 5,403,856, all of which are incorporated herein by reference in their entirety. The preparation of ramipril has also been described in EP 0 079 022 A2 and EP 0 317 878 A1, which are incorporated herein by reference in their entirety. It should be noted that the methods and compositions provided herein, when referring to ramipril are intended to encompass the use of ramipril and pharmaceutically acceptable salts thereof.

### Methods of Treatment

Described herein are methods of lowering glucose levels by administering a therapeutic amount of ramipril or a pharmaceutically acceptable salt thereof. For example, the methods described herein include a method of lowering glucose levels in a patient by administering to a patient diagnosed with dysglycemia, a therapeutically effective amount of ramipril or a pharmaceutically acceptable salt thereof for a sufficient period of time to reduce the glucose levels in said patient.

Patients diagnosed with dysglycemia include patients diagnosed with diabetes, and patients with pre-diabetic glucose levels such as patients diagnosed with impaired fasting glucose and impaired glucose tolerance. In certain embodiments of the methods described herein, dysglycemic patients can include individuals with no history of cardiovascular disease. Alternatively, in certain embodiments of the methods described herein, dysglycemic patients can include individuals with a history of cardiovascular disease.

In certain embodiments, also described herein are methods of lowering glucose levels in a patient comprising administering to a patient with impaired glucose tolerance or impaired fasting glucose, or both impaired glucose tolerance and impaired fasting glucose, a therapeutically effective amount of ramipril for a sufficient period of time to reduce the glucose levels in said patient. In other embodiments, also described herein are methods of lowering glucose levels in a patient comprising administering to a patient with diabetes, a therapeutically effective amount of ramipril for a sufficient period of time to reduce the glucose levels in said patient.

In the methods of lowering glucose levels described above, the glucose levels can include fasting plasma glucose levels or two hour post-load glucose levels or both fasting plasma glucose levels and two hour post-load glucose levels. For example, described herein are methods of lowering fasting plasma glucose levels by administering to a patient with dysglycemia, a therapeutically effective amount of ramipril for a sufficient period of time to reduce fasting plasma glucose levels in said patient. Also described herein are methods of lowering fasting plasma glucose levels by administering to a patient diagnosed with impaired glucose tolerance, impaired fasting glucose, both impaired glucose tolerance and impaired fasting glucose or diabetes, a therapeutically effective amount of ramipril for a sufficient period of time to reduce fasting plasma glucose levels in said patient. Such patients can also have no history of cardiovascular disease.

Additionally, described herein are methods of lowering post-load glucose levels by administering to a patient with dysglycemia, a therapeutically effective amount of ramipril for a sufficient period of time to lower the post-load glucose level of said patient. For example, described herein are methods of lowering post-load glucose levels by administering to a patient diagnosed with impaired glucose tolerance, impaired fasting glucose, both impaired glucose tolerance and impaired fasting glucose or diabetes, a therapeutically effective amount of ramipril for a sufficient period of time to lower the post-load glucose level of said patient. Such patients can also have no history of cardiovascular disease.

Moreover, in certain embodiments described herein, glucose levels are lowered to normal glucose levels. For example, fasting plasma glucose levels can be lowered to normal fasting plasma levels and two hour post-load glucose levels can be lowered to normal two hour post-load glucose levels.

Also described herein are methods of lowering glucose levels in a patient comprising administering to a patient with diabetes and no history of cardiovascular disease, a therapeutically effective amount of ramipril or a pharmaceutically acceptable salt thereof for a sufficient period of time to reduce the glucose levels in said patient. In such embodiments, diabetic glucose levels can be lowered to impaired fasting glucose levels or impaired glucose tolerance levels. Alternatively, diabetic glucose levels can be lowered to normal fasting glucose levels or normal glucose tolerance levels.

The methods described herein also include methods of preventing or reducing the frequency of a disorder associated with elevated glucose levels comprising administering to a patient diagnosed with dysglycemia, a therapeutically effective amount of ramipril or a pharmaceutically acceptable salt thereof for a sufficient period of time to prevent or reduce the frequency of said disorder. Such patients can include individuals with or diagnosed with diabetes, impaired glucose tolerance or impaired fasting glucose, or both impaired glucose tolerance and impaired fasting glucose. Additionally, dysglycemic patients can include individuals with no history of cardiovascular disease or individuals with a history of cardiovascular disease. For example, described herein are methods of preventing or reducing the frequency of a disorder associated with elevated glucose levels comprising administering to a patient diagnosed with impaired glucose tolerance or impaired fasting glucose, or both impaired glucose tolerance and impaired fasting glucose, a therapeutically effective amount of ramipril or a pharmaceutically acceptable salt thereof for a sufficient period of time to prevent or reduce the frequency of said disorder.

Additionally, described herein are methods of preventing or reducing the frequency of a disorder associated with elevated glucose levels comprising administering to a patient with diabetes and no history of cardiovascular disease, a therapeutically effective amount of ramipril or a pharmaceutically acceptable salt thereof for a sufficient period of time to prevent or reduce the frequency of said disorder.

Such disorders can include cardiovascular events, renal events, eye complications, and amputation. Examples of cardiovascular events include, but are not limited to, myocardial infarction, stroke, cardiovascular related death, heart failure, angina, revascularization, ventricular arrhythmia, acute congenital heart disease ischemia or atrial tachyarrhythmia. Examples of renal events include, but are not limited to, nephropathy or renal failure.

Other disorders include liver inflammation. Liver inflammation can be measured by ALT levels. As discussed above, ALT is a transaminase enzyme that is usually found in the liver and heart cells. Increased ALT levels in blood plasma may be an indication of inflammation of the liver or other liver dysfunctions as well as increased whole-body insulin sensitivity and/or insulin secretion. In certain embodiments described herein, decreasing the occurrence of liver inflammation can be accomplished by the reduction of ALT levels by administering a therapeutically effective amount of ramipril to a patient in need thereof. For example, described herein are methods of lowering ALT levels in a patient comprising administering to a patient with dysglycemia such as, diabetes, impaired glucose tolerance or impaired fasting glucose, or both impaired glucose tolerance and impaired fasting glucose, a therapeutically effective amount of ramipril for a sufficient period of time to reduce ALT levels in said patient. Such dysglycemic patients can include individuals with no history of cardiovascular disease or, alternatively, patients with a history of cardiovascular disease.

Also described herein are methods of preventing diabetes by administering to a patient diagnosed with dysglycemia and whose glucose levels have not reached diabetic levels, a therapeutically effective amount of ramipril for a period of three years or longer. Such dysglycemic patients include patients diagnosed with impaired fasting glucose and impaired glucose tolerance. Also, such dysglycemic patients can include patients with no history of cardiovascular disease or, alternatively, patients with a history of cardiovascular disease. For example, described herein are methods of preventing diabetes by administering to a patient diagnosed with impaired glucose tolerance or impaired fasting glucose, or both impaired glucose tolerance and impaired fasting glucose, a therapeutically effective amount of ramipril for a period of three years or longer.

Also, described herein are methods of preventing diabetes by administering to a patient with normal glucose levels such as, normal fasting glucose levels and normal post-load glucose levels, a therapeutically effective amount of ramipril for a sufficient period of time to prevent diabetes in said patient. In certain embodiments, a therapeutically effective amount of ramipril can be administered for period of three years, four years, five years, six years, seven years, eight years, nine years, ten years, fifteen years, twenty years or longer. For example, the therapeutically effective amount of ramipril can be administered for period of more than five years.

Additionally, described herein are methods of delaying the onset of diabetes in a patient comprising administering to a patient diagnosed with dysglycemia and whose glucose levels have not reached diabetic levels, a therapeutically effective amount of ramipril for a period of three years or more. Such dysglycemic patients include patients diagnosed with impaired fasting glucose and impaired glucose tolerance. Also, such dysglycemic patients can include patients with no history of cardiovascular disease or, alternatively, patients with a history of cardiovascular disease. For example, described herein are methods of delaying the onset of diabetes in a patient comprising administering to a patient diagnosed with impaired glucose tolerance or impaired fasting glucose, or both impaired glucose tolerance and impaired fasting glucose, a therapeutically effective amount of ramipril for a period of three years or more.

Also, described herein are methods of delaying the onset of diabetes by administering to a patient with normal glucose levels such as, normal fasting glucose levels and normal post-load glucose levels, a therapeutically effective amount of ramipril for a sufficient period of time to delay the onset of diabetes in said patient. In certain embodiments, a therapeutically effective amount of ramipril can be administered for period of three years, four years, five years, six years, seven years, eight years, nine years, ten years, fifteen years, twenty years or longer. For example, the therapeutically effective amount of ramipril can be administered for period of more than five years.

### Compositions

Ramipril used in the methods provided herein can be incorporated in any composition (e.g., pharmaceutical composition) known in the art. Suitable pharmaceutical composition can include individualized or multi unit doses, such as tablets, caplets, powders, suspension tablets, chewable tablets, rapid melt tablets, capsules, e.g., a single or double shell gelatin capsule, tablet-filled capsules, effervescent powders, effervescent tablets, pellets, granules, liquids, solutions, or suspensions, respectively. Of special interest are tablets, caplets and capsules.

Ramipril suitable for the methods provided herein can be any form of ramipril known in the art including, but not limited to, uncoated or can be coated with a coat forming material.

Uncoated ramipril suitable for the methods provided herein includes ramipril, as obtained from the Sanofi-Aventis Deutschland GmbH (Frankfurt on Main, Germany). Coated ramipril suitable for the methods provided herein can be any coated ramipril known in the art. For example, coated ramipril suitable for the methods provided herein can include ramipril particles that are coated with a suitable coat forming material. Coated ramipril suitable for the methods provided herein can be partially, substantially or completely covered with a coat forming material. Ramipril particles can include, but are not limited to, coated ramipril micro- or nanoparticles, coated ramipril crystalline particles, coated individual ramipril crystals and coated ramipril agglomerates, granules or beads. One suitable type of ramipril agglomerates is the GEcoated ramipril agglomerates, manufactured by Sanofi-Aventis Deutschland GmbH (Frankfurt on Main, Germany). Such GEcoated ramipril agglomerates are ramipril agglomerates coated with a hydroxypropyl methylcellulose polymer coating (1.192 mg GEcoated granules=1.0 mg ramipril). Coated ramipril particles suitable for the methods provided herein can also be made according to the methods disclosed in U.S. Pat. Nos. 5,061,722; 5,151,433; 5,403,856; and 5,442,008, U.S. Provisional Application No. 60/625,270 and a Co-pending U.S. Application Publication Nos. 20060134213 and 20060159742, incorporated by reference herein in their entireties. Compositions useful in the methods provided herein can also contain anhydrous, pharmaceutical grade ramipril powder comprising coated ramipril particles.

In certain embodiments, the pharmaceutical compositions useful in the methods provided herein comprise ramipril wherein the ramipril is substantially stable against decomposition into degradant products, such as ramipril-diacid and ramipril-DKP. Additionally, the ramipril compositions useful in the methods provided herein can have improved stability and shelf-life. This improved stability allows the ramipril compositions to maintain potency and improve effectiveness and bioavailability of ramipril compared to other ramipril formulations.

Stabilized ramipril compositions, including methods for their preparation, useful in the methods provided herein are described in U.S. Patent Application Publication No. 2006/0134213 A1, the contents of which is incorporated by reference herein in its entirety.

In one embodiment, the pharmaceutical compositions useful in the methods provided herein have a rate of decomposition of ramipril of less than about 0.04% to about 0.095% of the total weight of ramipril at room temperature, on average per month for at least about 36 months from the date that the ramipril compositions are first formulated. Certain suitable pharmaceutical compositions have ramipril-DKP formation of less than about 0.04% to about 0.085% of the total weight of ramipril at room temperature, on average per month for an extended period, have ramipril-DKP formation of less than about 0.04% to about 0.055% of the total weight of ramipril at room temperature, per month on average for such an extended period, or have ramipril-DKP formation of less than about 0.04% to about 0.042% of the total weight of ramipril at room temperature, per month on average for an extended period of time.

The ramipril compositions useful in the methods provided herein can result in ramipril-DKP formation of less than about 0.3% during about the first three months of the total weight of ramipril and less than about 2.0% of the total weight of ramipril during a period of at least about 36 months after the first three month period. Ramipril compositions useful in the methods provided herein can result in ramipril-DKP formation of less than about 0.3% of the total weight of ramipril during about the first three months and less than about 1.5% of the total weight of ramipril during a period of at least about 36 months after the first three month period.

In one embodiment, the compositions useful in the methods provided herein comprise ramipril, wherein the rate of ramipril decomposition to ramipril-DKP, is less than about 0.3% of the total weight of the ramipril during about the first three months after the compositions are formed.

In another embodiment, the compositions useful in the methods provided herein comprise ramipril, wherein the rate of ramipril decomposition to ramipril-DKP, is less than about 0.75% of the total weight of the ramipril during about the first 6 months after the compositions are formed.

In yet another embodiment, the compositions useful in the methods provided herein comprise ramipril, wherein the rate ramipril decomposition to ramipril-DKP, is less than about 3.0% of the total weight of the ramipril during about the first 36 months after the compositions are formed.

Pharmaceutical compositions useful in the methods provided herein can also include pharmaceutically acceptable additives into any suitable type of unit dosage form. Suitable additives include, but are not limited to, blending agents, diluents, binders, vehicles, carriers, excipients, binders, disintegrating agents, lubricants, swelling agents, solubilizing agents, wicking agents, cooling agents, preservatives, stabilizers, sweeteners, flavors, polymers, etc.

The blending agent can be any substance suitable for pre-blending and co-milling, which stabilizes the drug and significantly reduces the degradation of the drug. The phrase “blending agent” is interchangeable with “blending compound”. The blending agent can coat the ramipril and reduce the degradation rate.

Blending agents contemplated herein include polymers, starches, stearates, silicas, waxes (atomized glyceryl palmitostearate, dioctyl sodium sulphosuccinate), surfactants, and fatty acids (in one embodiment having a chain length of eight carbons or greater which may contain one or more double bonds). For example, blending agents suitable for the compositions useful in the methods provided herein include, but are not limited to, long chain fatty acid-containing glycerol esters. Blending agents include, but are not limited to, glyceryl behenate, glyceryl stearate, stearyl alcohol, magnesium stearate, macrogol stearate ether, palmitostearate, ethylene glycol, polyethylene glycol, ethylene oxide polymers, sodium lauryl sulfate, magnesium lauryl sulfate, sodium oleate, sodium stearyl fumerate, leucine, stearic acid, cetyl alcohol, lauryl alcohol, amylopectin, poloxymer or combinations thereof. Most preferably, the blending agent is glyceryl behenate.

The blending agent can be present from at least about 0.1 wt % and above by weight of the total composition. In a specific embodiment, the blending agent is present at about 0.5 wt. % and above. In another specific embodiment, the blending agent is present at about 1.0 wt. % and above. In another specific embodiment, the blending agent is present at about 2.0 wt. % and above. In a specific and preferred embodiment, the blending agent is present at about 3.0 wt. % and above. In another specific embodiment, the blending agent is present at about 4.0 wt. % and above (e.g., 5 and 10 wt. %).

The blending agent can be present from at least 0.1 wt % and above by weight of the total composition. In a specific embodiment, the blending agent is present at 0.5 wt. % and above. In another specific embodiment, the blending agent is present at 1.0 wt. % and above. In another specific embodiment, the blending agent is present at 2.0 wt. % and above. In a specific and preferred embodiment, the blending agent is present at 3.0 wt. % and above. In another specific embodiment, the blending agent is present at 4.0 wt. % and above (e.g., 5 and 10 wt. %).

Additionally, the blending agent can be present in a ratio of about 1:10 to about 10:1 of ramipril. The blending agent can be present in a ratio of about 1:5 to about 5:1 or about 1:2 or 2:1 of ramipril.

In yet another embodiment, the pharmaceutical compositions useful in the methods provided herein comprise ramipril and a blending agent, wherein ramipril is coated by the blending agent. Ramipril can be substantially coated by the blending agent. Ramipril is substantially coated when the blending agent coats ramipril wherein ramipril has a low or no rate of degradation. For example, ramipril can be between about 50% to 100%, about 75% to 100%, about 85% to 100%, or about 95% to 100% coated by the blending agent.

Examples of excipients include, but are not limited to, acacia, alginic acid, croscarmellose, gelatin, gelatin hydrosylate, mannitol, plasdone, sodium starch glycolate, sorbitol, sucrose, and xylitol. For molded or compressed tablet formulations, suitable excipients that may be used include amorphous lactose, beta lactose, microcrystalline cellulose, croscarmellose sodium, dicalcium phosphate, carboxymethyl cellulose, hydroxypropyl cellulose, polyethylene gylcols, sodium lauryl sulfate, and the like.

Examples of additional stabilizers or preservatives include, but are not limited to, parahydroxybenzoic acid alkyl esters, antioxidants, antifungal agents, and other stabilizers/preservatives known in the art.

Examples of coloring agents include, but are not limited to, water soluble dye, Aluminum Lake, ion oxide, natural colors, titanium oxide, and the like.

Examples of diluents or fillers include, but are not limited to, water-soluble and/or water-insoluble tabletting fillers. The water-soluble diluent agent may be constituted from a polyol of less than 13 carbon atoms, in the form of directly compressible material (the mean particle size being between about 100 and about 500 microns), in the form of a powder (the mean particle size being less than about 100 microns) or a mixture thereof. The polyol is preferably chosen from the group comprising of mannitol, xylitol, sorbitol and maltitol. The water-insoluble diluent agent may be a cellulosic derivative, such as, microcrystalline cellulose or a starch, such as, pre-gelatinized starch. Especially preferred diluents are those with minimal moisture content, such as lactose monohydrate and magnesium oxide.

Examples of disintegrating agents include, but are not limited to, cross-linked sodium carboxymethylcellulose, crospovidone and their mixtures. A part of the disintegrating agent may be used for the preparation of PPI, cholinergic agonist, parietal activator and/or antacid granules.

Examples of lubricating agents include, but are not limited to, magnesium stearate, stearic acid and its pharmaceutically acceptable alkali metal salts, sodium stearyl fumerate, Macrogol 6000, glyceryl behenate, talc, colloidal silicon dioxide, calcium stearate, sodium stearate, Cab-O-Sil, Syloid, sodium lauryl sulfate, sodium chloride, magnesium lauryl sulfate, talc and their mixtures. A portion of the lubricant may be used as an internal solid lubricant which is blended and granulated with other components of the granulation. Another portion of the lubricant may be added into the final blended material just before compression or encapsulation that coats the outside of the granules in the final blend.

Examples of swelling agents include, but are not limited to, starches; polymers; cellulosic materials, such as, microcrystalline cellulose, hydroxypropylmethyl cellulose, sodium carboxymethylcellulose and ethyl cellulose; waxes such as bees wax; natural materials, such as, gums and gelatins; or mixtures of any of the above.

Examples of polymers include, but are not limited to, polysaccharides, celluloses, and organic moieties such as polyvinyl pyrrolidines and plastics.

Examples of celluloses include, but are not limited to, hydroxypropylcellulose, hydroxypropylmethylcellulose, hydroxylpropyl-methylcellulose, hydroxyethylcellulose, ethylcellulose, cellulose acetate phthalate, cellulose acetate, polyvinyl acetate phthalate, polyvinylpyrrolidone, gelatin, hydroxypropyl methyl cellulose acetate succinate, hydroxypropyl methyl cellulose succinate, hydroxylpropyl cellulose acetate succinate, hydroxyethyl methyl cellulose succinate, hydroxyethyl cellulose acetate succinate, hydroxypropyl methyl cellulose phthalate, hydroxethyl methyl cellulose acetate succinate, hydroxyethyl methyl cellulose acetate phthalate, carboxyethyl cellulose, carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methylcellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimelllitate succinate, cellulose propionate trimellitate, cellulose butryrate trimellitate, cellulose acetate terephthalate, cellulose acetate isophthalate, cellulose acetate pyridine dicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid, cellulose acetate, ethyl picolinic acid cellulose acetate.

Other polymers that may be suitable for use with the present compositions useful in the methods provided herein include, but are not limited to, acrylate and methacrylate copolymers. Exemplary commercial grades of such copolymers include the EUDRAGIT® series, which are copolymers of methacrylates, acrylates, carboxylic acid-functionalized vinyl polymers, such as the carboxylic acid functionalized polymethacrylates and carboxylic acid functionalized polyacrylates, amine-functionalized polyacrylates and polymethacrylates; proteins such as gelatin and albumin, and carboxylic acid functionalized starches such as starch glycolate, carboxylic acid functionalized polymethyacrylates, carboxylic acid functionalized polyacrylate, amine-functionalized polyacrylates, amine-functionalized polymethacrylates, proteins, carboxylic acid functionalized starches, vinyl polymers and copolymers having at least one substituent selected from the group consisting of hydroxyl, alkylacyloxy, and cyclicamido; polyvinyl alcohols that have at least a portion of their repeat units in the unhydrolyzed (vinyl acetate) form; polyvinyl alcohol polyvinyl acetate copolymers; polyvinyl pyrrolidone; polyethylene polyvinyl alcohol copolymers, polyoxyethylene-polyoxypropylene copolymers, alkylacyloxy-containing repeat units, or cyclicamido-containing repeat units; polyvinyl alcohols that have at least a portion of their repeat units in the unhydrolyzed form; polyvinyl alcohol polyvinyl acetate copolymers; polyethylene glycol, polyethylene glycol polypropylene glycol copolymers, polyvinyl pyrrolidone polyethylene polyvinyl alcohol copolymers, and polyoxyethylene-polyoxypropylene block copolymers.

The flavouring may be advantageously chosen to give a combination of fast onset and long-lasting sweet taste and get a “round feeling” in the mouth with different texturers or additives. Cooling agents can also be added in order to improve the mouth feeling and provide a synergy with flavors and sweetness. Various other materials may be present as coatings or to otherwise modify the physical form of the dosage unit. For instance, tablets or capsules may be coated with shellac, sugar or both.

### Administration

Ramipril used in the methods and compositions provided herein can be administered in a dose that will achieve a therapeutic effect. For example ramipril can be administered in an amount between about 0.0001 mg/day to 1000 mg/day. In certain embodiments ramipril is administered between about 0.001 mg/day to 750 mg/day, or between about 0.01 mg/day to 500 mg/day, or between about 0.1 mg/day to 250 mg/day, or between about 0.1 mg/day to 100 mg/day, or between about 1.25 mg/day to 50 mg/day, or between about 1.25 mg/day to 20 mg/day. In certain embodiments, ramipril is administered in an amount of 1.25 mg/day, 2.5 mg/day, 5 mg/day, 10 mg/day, 15 mg/day or 20 mg/day.

Ramipril can also be administered in an amount between about 0.000001 mg/kg/day to 15 mg/kg/day. In certain embodiments ramipril is administered between about 0.00001 mg/day to 10 mg/kg/day, or between about 0.0001 mg/day to 5 mg/kg/day, or between about 0.001 mg/kg/day to 3 mg/kg/day, or between about 0.01 mg/kg/day to 1 mg/kg/day.

Ramipril used in the methods provided herein can be administered by any means known in the art. Suitable routes of administration include parenteral (e.g., subcutaneous, intramuscular, intraorbital, intracapsular, intraspinal, intrasternal, intravenous, intradermal, intraperitoneal, intraportal, intra-arterial, intrathecal, transmucosal, intra-articular, and intrapleural,), transdermal (i.e., topical), epidural, mucosal (e.g., intranasal) injection or infusion, as well as oral, inhalation, pulmonary, and rectal administration. Oral administration can be accomplished, for example, by administering to the patient a solid or liquid oral dosage form by mouth or via a gastric feeding tube, a duodenal feeding tube, a nasogastric (ng) tube, a gastrostomy, or other indwelling tubes placed in the GI tract.

Depending on the mode of administration ramipril can be incorporated in suppositories, suspensions, liquids, powders, creams, transdermal patches, and depots. Oral pharmaceutical compositions useful in the methods provided herein are generally in the form of individualized or multi unit doses, such as tablets, caplets, powders, suspension tablets, chewable tablets, rapid melt tablets, capsules, e.g., a single or double shell gelatin capsule, tablet-filled capsules, effervescent powders, effervescent tablets, pellets, granules, liquids, solutions, or suspensions, respectively.

When the pharmaceutical compositions useful in the methods provided herein are formed into tablets or caplets, it is to be understood that the tablets or caplets can be scored, and that they may be of any suitable shape and size, such as round, square, rectangular, oval, diamond, pentagon, hexagon or triangular, so long as the objectives of the methods provided herein are not defeated. It is to be further understood that when tablet-filled capsules are selected, the tablets utilized therewith can be formed into shapes that either (a) correspond to the capsules to permit over-coating or encapsulation via the capsules or (b) readily fit inside the capsules.

The oral pharmaceutical compositions can contain ramipril in any therapeutically effective amount, such as from about 0.001 mg or less to about 200 mg or more, or preferably from about 0.01 mg to about 100 mg or preferably from about 0.1 mg to about 50 mg. In one embodiment, the dosage range will be between about 1.25 mg to about 20 mg per patient per day.

By way of example, oral unit dosage forms or compositions useful in the methods provided herein can contain ramipril in an amount of about 1.25 mg, about 2.5 mg, about 5 mg, about 7.5 mg, about 10 mg, 12.5 mg, about 15 mg, about 20 mg, about 25 mg, about 30 mg, about 40 mg, about 50 mg, about 60 mg, about 70 mg, about 75 mg, about 80 mg, about 90 mg, or about 100 mg. Of course, it should be appreciated that a particular unit dosage form and amount can be selected to accommodate the desired frequency of administration used to achieve a specified daily dosage and therapeutic effect.

Of particular interest are 1.25, 2.5, 5, 10, 15 and 20 mg ramipril tablets, 1.25, 2.5, 5, 10, 15 and 20 mg ramipril caplets, 1.25, 2.5, 5, 10, 15 and 20 mg ramipril capsules and 1.25, 2.5, 5, 10, 15 and 20 mg ramipril tablet-filled capsules.

Consistent with the present methods, these and other dosage forms discussed herein can be administered to patients on a regimen of one, two or more doses per day, at any time of the day.

In accordance with the methods described herein, ramipril can be administered over a period of a single day or multiple days. In certain embodiments, ramipril can be administered for a period of one week, two weeks, three weeks, four weeks or longer. In other embodiments, ramipril can be administered for a period of one month, two months, three months, four months, five months, six months, seven months, eight months, nine months, ten months, eleven months or longer. In still other embodiments, ramipril can be administered for a period of one year, two years, three years, four years, five years, six years, seven years, eight years, nine years, ten years, fifteen years, twenty years or longer.

The dosage of ramipril in the compositions useful in the methods provided herein can be varied. Ramipril can be administered to a patient in need of treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets being followed by a patient, concurrent medication, and other factors, recognized by those skilled in the art. Based upon the foregoing, precise dosages depend on the condition of the patient and are determined by discretion of a skilled clinician. Generally, ramipril daily dosage levels of between about 0.010 to about 1.5 mg/kg of body weight are administered daily to mammalian patients, e.g., humans having a body weight of about 70 kg. The ramipril dosage range will generally be about 1.25 mg to 50 mg per patient per day, administered in single or multiple doses.

Nonetheless, it should be understood that safe and effective amounts of ramipril utilized in accordance with the present methods will vary with the particular condition and/or symptoms being treated, the age, weight and physical conditions of the patient being treated, the severity of the condition and/or symptoms, the duration of treatments, the nature of concurrent therapies, the specific dosage form employed, the particular pharmaceutically acceptable carriers utilized, and like factors within the knowledge and expertise of the attending physicians. Exemplary safe and effective amounts of ramipril include those amounts mentioned herein, administered one or more times per day.

### EXAMPLES

DREAM Clinical Trial

Methods

DREAM is a large, international, multi-centre, randomized double-blind placebo-controlled trial that determined whether the angiotensin converting enzyme-inhibitor ramipril or the thiazolidinedione (TZD) rosiglitazone prevent the development of diabetes mellitus, as well as, whether ramipril causes a regression of glucose levels to normal glucose levels in patients with elevated glucose levels.

A total of 5269 participants with IGT or IFG, or both IGT and IFG were recruited. Participants were followed for an average of 3 years after randomization. Participants were enrolled between July 2001 and August 2003. All eligible participants were 30 years of age or older and had IGT and/or IFG. Participants had no history of diabetes (except gestational), cardiovascular disease or intolerance to ACE-inhibitors or TZDs. The exclusion criteria is shown in Table 1.

TABLE 1

| Criteria | Definition of Criteria | | |
|---|---|---|---|
| Drug Use | a) | current use of ACE-I and/or TZDs and inability to discontinue these | |
| | | medications | |
| | b) | known hypersensitivity to ACE-I | |
| | c) | prior use of antidiabetic medications except during pregnancy | |
| | d) | use of systemic glucocorticoids or niacin | |
| Cardiovascular | a) | ejection fraction known to be <40% or congestive heart failure, or | |
| Disease | | existing clinical CV disease (previous MI or stroke; angina with | |
| | | either >50% stenosis in >=2 major coronary arteries, or ST | |
| | | depression of >=2 mm, or a positive nuclear test, previous coronary | |
| | | angioplasty, stent or bypass; previous limb bypass or vessel | |
| | | angioplasty or angiographic evidence of >50% stenosis, or | |
| | | intermittent claudication with an ankle/arm pressure <=0.8) | |
| | b) | uncontrolled hypertension requiring ACE inhibitors or angiotensin 2 | |
| | | receptor blockers | |
| Other Criteria | a) | History of diabetes (except gestational DIABETES MELLITUS) or | |
| | | on antidiabetic medication | |
| | b) | Renal or Hepatic Disease | |
| | | i) | renal artery stenosis |
| | | ii) | creatinine clearance <0.6 ml/s or serum creatinine ≧200 umol/l |
| | | iii) | clinical proteinuria (≧1+ proteinuria on dipstick or ≧300 mg of |
| | | | albuminuria/day) |
| | | iv) | measured alanine transferase (ALT) ≧2.5 times the upper limit of |
| | | | normal |
| | | v) | active liver disease including jaundice, chronic hepatitis, previous |
| | | | liver transplant |

| | |
|---|---|
| c) | Major illness with life expectancy <5 years or that may interfere |
| | with participation |
| d) | Use of another experimental drug |
| e) | Pregnant or unwilling to use reliable contraception (fertile women |
| | will have a pregnancy test prior to randomization) |
| f) | Major psychiatric disorder |
| g) | Diseases and medications that affect glucose tolerance (e.g. |
| | pheochromocytoma, Cushing's syndrome, acromegaly, steroid- |
| | dependent asthma, protease inhibitors, antipsychotics) |
| h) | Unwillingness to be randomized or sign informed consent |
| i) | Known uncontrolled substance abuse |
| i) | Inability to communicate with clinic staff |

Participants were recruited from a wide variety of sources including first degree relatives of diabetic people in diabetes mellitus clinics; ads in newspapers; pharmacies; national diabetes associations; newsletters; lipid, hypertension, cardiology, and family practice clinics; community announcements; screening programs in workplaces and other defined populations; public presentations; media stories; and targeted mailings using mailing list brokers.

Screening efforts were focused on individuals of different ages depending on their ethnic origins. Due to the fact that the prevalence of IGT, IFG and type 2 diabetes mellitus rises with age, screening efforts generally focused on individuals age 45 years or over. However, for individuals of South Asian, Aboriginal, Chinese, or Hispanic origin, in whom IGT, IFG and diabetes mellitus occur at younger ages, screening efforts targeted people age 30 years or over. These groups were targeted by publicizing the study within these communities and by direct mail campaigns.

Eligible participants were ether randomized to ramipril or placebo, or rosiglitazone or placebo using a 2×2 factorial design, using a concealed, computerized telephone randomization system. Participants who were given ramipril were started at 5 mg daily for the first two months, 10 mg at the second month visit and then 15 mg after one year. Participants who were given rosiglitazone were started at 4 mg daily for the first two months and then 8 mg after that.

Follow up visits occurred at 2 months (±1 week), 6 months (±4 weeks), and every 6 months (±4 weeks) thereafter. Blood was drawn for local measurement of ALT at 2, 4, 6, 8, 10 and 12 months to screen for any liver toxicity.

At each follow-up visit contact information was verified, and data regarding any hospitalization, adverse events and adherence was collected. Pulse and arm and ankle blood pressure were recorded annually. At the year 2 and “end” visit, concomitant medications, weight, waist and hip circumference and ECG were also recorded, and a first morning (or if unavailable, a random) urine and fasting blood sample were sent centrally for later assay of the albumin/creatinine ratio, FPG, HbA1c and for storage.

Diabetes mellitus was screened for at every annual visit. At the 2 year and final visit, a local FPG and 2 hr plasma glucose after an OGTT was done on every participant.

Results

A total of 24,592 participants were screened from 191 centers in 21 countries. Of those screened, 5,808 entered the run-in phase of the trial. The most common reasons for exclusion were lack of eligibility (94%) and participant refusal (3%). Of those entering the run-in phase, 5,269 participants were randomized (739 with IFG alone, 4,530 with IFG and/or IGT). The most common reasons for exclusion in run-in were ineligibility (n=287) and participant refusal (n=151).

At year one, 84% of participants randomized to ramipril and 88% randomized to placebo continued study medication. The corresponding proportions at two years were 79% and 83%, at three years 73% and 78% and at study end 73% and 78%. The most common reasons for discontinuation of medication were participant refusal (16.8% ramipril and 17.3% placebo), cough (9.8% ramipril and 1.8% placebo), physician's advice (2.2% ramipril and 2.4% placebo) and peripheral edema (0.9% ramipril and 1.0% placebo). Open label ACE-inhibitors or angiotensin receptor blockers were used in 2.6% of ramipril and 4.0% of placebo participants.

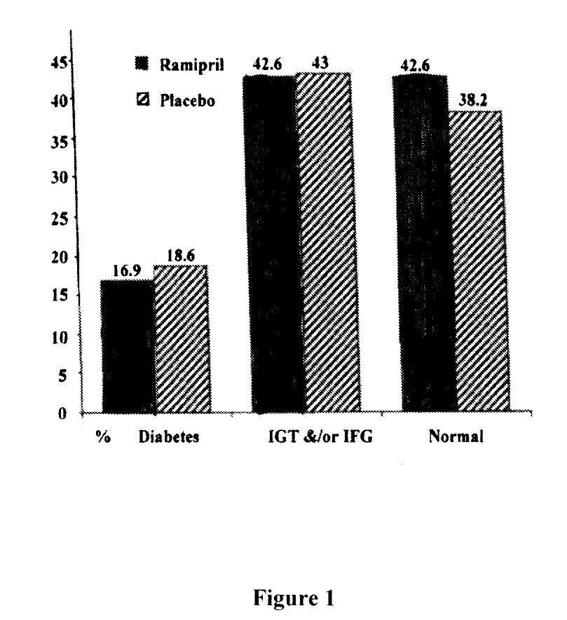

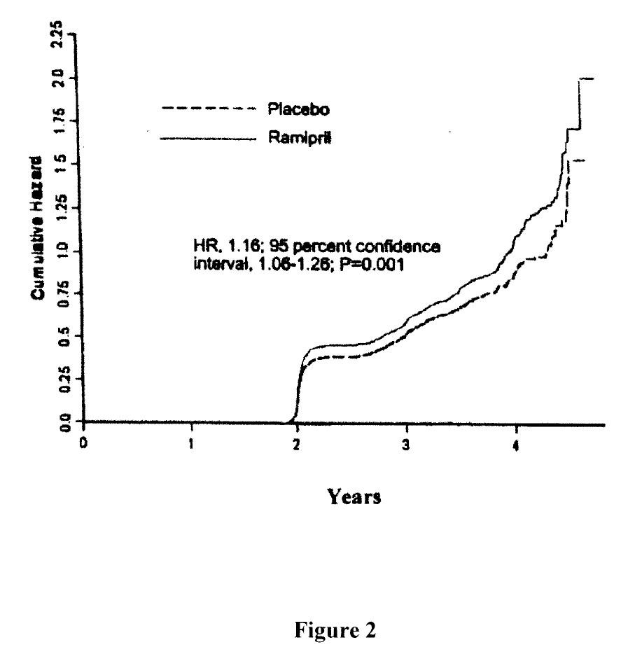

The status at study end of all participants in terms of diabetes, IFG, IGT, or normal glucose values is shown in FIG. 1. By study end, more individuals in the ramipril group achieved normal glucose levels in both fasting and two hour glucose levels compared to placebo (1117 (42.6%) versus 1011 (38.2%); hazard ratio, 1.16; 95% confidence interval, 1.06-1.26; P=0.001). The Kaplan-Meier estimates for regression are shown in FIG. 2. The two hour plasma glucose values were first re-measured at two years and therefore the results are presented from that time onwards.

The median fasting plasma glucose levels for the ramipril group and the placebo group were both 5.90 mmol/L. The median fasting plasma glucose levels for the ramipril group decreased to 5.52 at one year, 5.57 at two years and 5.63 at three years. The median fasting plasma glucose levels for the placebo group decreased to 5.6 at one year, 5.63 at two years and 5.7 at three years. The median fasting plasma glucose levels at the end of the study was 5.68 in the ramipril group versus 5.7 in the placebo group (P=0.04).

Median two hour post-load glucose values for participants in the ramipril group were 8.64, 7.13, 7.40, and 7.50 mmol/L at baseline, two years, three years and at the end of the study compared to 8.74, 7.35, 7.5, and 7.7 in the placebo group for the corresponding time periods (P=0.01).

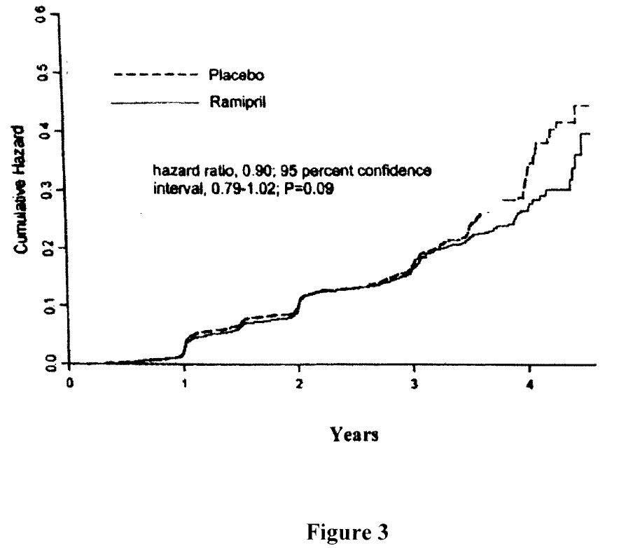

The diabetes development rates were similar in both groups until the third year after which time there was a tendency towards a lower rate in the ramipril group, as shown in FIG. 3. In the ramipril group 442 (16.9%) participants developed diabetes compared to 491 (18.6%) in the placebo group (hazard ratio, 0.89; 95% confidence interval, 0.78-1.01; P=0.08), as shown in FIG. 1.

Ramipril decreased ALT during the first year. The average ALT with ramipril was 25.6 U/l and 26.4 U/l with the placebo group (P=0.04). The baseline systolic blood pressure dropped from 136.2 mm Hg and 136.0 mm Hg in the ramipril and placebo groups respectively by 8.2 mm Hg with ramipril versus 3.9 mm Hg with placebo at two months (P<0.001). These differences persisted throughout follow-up. Baseline diastolic blood pressure was similar between both groups (83.4 mm Hg) dropping by 5.3 mm Hg in the ramipril group compared to 3.0 mm Hg in the placebo group (P<0.001).

While the foregoing description may represent preferred embodiments of the present invention, it should be understood that various additions, modifications, and substitutions may be made therein without departing from the spirit and scope of the present invention as defined in the accompanying claims. In particular, it will be clear to those skilled in the art that the present invention can be embodied in other specific forms, structures, arrangements, and proportions, and with other elements, materials, and components, without departing from the spirit or essential characteristics thereof. One skilled in the art will appreciate that the invention can be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims and not limited to the foregoing description. Furthermore, all references mentioned herein are incorporated by reference in their entirety for all purposes.

What is claimed:

1. A method of lowering glucose levels in a patient comprising administering to a patient diagnosed with dysglycemia, wherein the patient has no history of cardiovascular disease, a therapeutically effective amount of ramipril or a pharmaceutically acceptable salt thereof for a sufficient period of time to reduce the glucose levels in said patient.

2. The method of claim 1, wherein the patient has been diagnosed with diabetes.

3. The method of claim 1, wherein the glucose levels are fasting plasma glucose levels or two hour post-load glucose levels.

4. The method of claim 1, wherein the glucose levels are lowered to normal fasting plasma levels or lowered to normal two hour post-load glucose levels.

5. The method of claim 1, wherein the therapeutically effective amount of ramipril is between 1.25 mg/day to 20 mg/day.

6. The method of claim 1, wherein ramipril is administered in a capsule or a tablet.

7. A method of lowering glucose levels in a patient comprising administering to a patient with impaired glucose tolerance, impaired fasting glucose or both impaired glucose tolerance and impaired fasting glucose a therapeutically effective amount of ramipril or a pharmaceutically acceptable salt thereof for a sufficient period of time to reduce the glucose levels in said patient.

8. A method of reducing the frequency of or preventing a disorder associated with elevated glucose levels comprising administering to a patient diagnosed with dysglycemia, wherein the patient has no history of cardiovascular disease, a therapeutically effective amount of ramipril or a pharmaceutically acceptable salt thereof for a sufficient period of time to prevent or reduce the frequency of said disorder.

9. The method of claim 8, wherein the patient has been diagnosed with diabetes.

10. The method of claim 8, wherein the patient has been diagnosed with impaired glucose tolerance, impaired fasting glucose or both impaired glucose tolerance and impaired fasting glucose.

11. The method of claim 8, wherein the therapeutically effective amount of ramipril is between 1.25 mg/day to 20 mg/day.

12. The method of claim 8, wherein ramipril is administered in a capsule or a tablet.

13. The method of claim 8, wherein the disorder is a cardiovascular event renal event, or liver inflammation.

14. The method of claim 13, wherein the cardiovascular event is myocardial infarction, stroke, cardiovascular related death, heart failure, angina, revascularization, ventricular arrhythmia, acute congenital heart disease ischemia or atrial tachyarrhythmia.

15. The method of claim 13, wherein the renal event is nephropathy or renal failure.

16. The method of claim 8, wherein the disorder is an eye complication or amputation.

17. The method of claim 13, wherein the liver inflammation is diagnosed by measuring ALT levels.

18. The method of claim 17, wherein the ALT levels are reduced.

19. A method of lowering ALT levels in a patient comprising administering to a patient with dysglycemia, such as, diabetes, impaired glucose tolerance, impaired fasting glucose or both impaired glucose tolerance and impaired fasting glucose, a therapeutically effective amount of ramipril or a pharmaceutically acceptable salt thereof for a sufficient period of time to reduce ALT levels in said patient.

20. The method of claim 19, wherein the patient has no history of cardiovascular disease.

21. A method of preventing diabetes or at least delaying the onset of diabetes in a patient comprising administering to a patient diagnosed with dysglycemia, wherein the glucose levels of the dysglycemic patient are lower than diabetic glucose levels, a therapeutically effective amount of ramipril for a period of five years or more.

22. The method of claim 21, wherein the patient has no history of cardiovascular disease.

23. The method of claim 21, wherein the patient has impaired glucose tolerance or impaired fasting glucose or both impaired glucose tolerance and impaired fasting glucose.

24. The method of claim 21, wherein the therapeutically effective amount of ramipril is between 1.25 mg/day to 20 mg/day.

25. The method of claim 21, wherein ramipril is administered in a capsule or a tablet.
